(19) United States
(12) Patent Application Publication
(10) Pub. No.: US 2007/0048349 A1
(43) Pub. Date: Mar. 1, 2007

(54) Surface-modified medical devices and methods of making

(75) Inventors: Joseph C. Salamone, Fairport, NY (US); Jay F. Kunzler, Canandaigua, NY (US); Joseph A. McGee, Dewitt, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/214,175

(22) Filed: Aug. 29, 2005

(51) Int. Cl. A61F 2/02

(52) U.S. Cl. 424/423

(57) ABSTRACT

A surface-modified medical device comprises a hydrophilic coating that comprises units of a hydrophilic monomer and a coupling agent that has coupling functional groups, at least a portion of which is coupled to the surface functional groups of the medical device. A method for producing such a surface-modified medical device comprises forming the hydrophilic coating by coupling units of the hydrophilic monomer to the medical device through the coupling agent. Medical devices, such as stents, implants, catheters, and ophthalmic devices, are included.

### BACKGROUND OF THE INVENTION

The present invention relates to medical devices having modified surfaces and method for making such devices. In particular, the present invention relates to ophthalmic devices having surfaces modified for increased surface hydrophilicity.

Advances in chemistry of materials for medical devices have increased their compatibility with a body environment and the comfort for their extended use therein. Furthermore, extended use of medical devices, such as ophthalmic lenses, has become increasingly favored due to the availability of soft contact lenses having high oxygen permeability (e.g., exhibiting high Dk values greater than 80) and/or high water content. Such lenses are increasingly made of silicone-containing materials. Although these materials have some desirable properties for ophthalmic applications, they tend to have relatively hydrophobic surfaces that have a high affinity for lipids and proteins. Accumulation of these materials can interfere with the clarity of the lens and the comfort of the wearer. In addition, hydrophobic surfaces tend to facilitate bacterial attachment thereto and growth thereon. Bacterial attachment to biomaterial surfaces is believed to be a contributing factor in device-related infection.

Thus, those skilled in the art have long recognized the need for modifying the surface of hydrophobic contact lenses, such as those comprising silicone, so that they are compatible with the eye. It is known that increased hydrophilicity of the contact lens surface improves the wettability of the contact lenses and decreases their susceptibility to deposition, particularly the deposition of proteins and lipids from the tear fluid during lens wear. Increased hydrophilicity, in turn, results in improved wear comfort of contact lenses. In the case of extended-wear lenses (i.e., lenses used without daily removal of the lens before sleep), the surface is especially important, since extended-wear lenses must be designed for high standards of comfort and biocompatibility over an extended period of time.

Silicone lenses have been subjected to plasma surface treatment to improve their surface properties; e.g., surfaces have been rendered more hydrophilic, deposit-resistant, scratch-resistant, or otherwise modified. Examples of previously disclosed plasma surface treatments include subjecting contact lens surfaces to a plasma comprising an inert gas or oxygen (see, for example, U.S. Pat. Nos. 4,055,378; 4,122,942; and 4,214,014); various hydrocarbon monomers (see, for example, U.S. Pat. No. 4,143,949); and combinations of oxidizing agents and hydrocarbons, such as water and ethanol (see, for example, WO 95/04609 and U.S. Pat. No. 4,632,844). U.S. Pat. No. 4,312,575 to Peyman et al. discloses a process for providing a barrier coating on a silicone or polyurethane lens by subjecting the lens to an electrical glow discharge (plasma) process conducted by first subjecting the lens to a hydrocarbon atmosphere followed by subjecting the lens to oxygen during glow discharge, thereby increasing the hydrophilicity of the lens surface.

U.S. Pat. Nos. 4,168,112; 4,321,261; and 4,436,730, all issued to Ellis et al., disclose methods for treating a charged contact lens surface with an oppositely charged ionic polymer to form a polyelectrolyte complex on the lens surface that improves wettability.

U.S. Pat. No. 4,287,175 to Katz discloses a method of wetting a contact lens that comprises inserting a water-soluble solid polymer into the cul-de-sac of the eye. The disclosed polymers include cellulose derivatives, acrylates and natural products such as gelatin, pectins, and starch derivatives.

U.S. Pat. No. 5,397,848 to Yang et al. discloses a method of incorporating hydrophilic constituents into silicone polymer materials for use in contact and intraocular lenses.

U.S. Pat. Nos. 5,700,559 and 5,807,636, both to Sheu et al., discloses hydrophilic articles (for example, contact lenses) comprising a substrate, an ionic polymeric layer on the substrate and a disordered polyelectrolyte coating ionically bonded to the polymeric layer.

U.S. Pat. No. 5,705,583 to Bowers et al. discloses biocompatible polymeric surface coatings. The polymeric surface coatings disclosed include coatings synthesized from monomers bearing a center of positive charge, including cationic and zwitterionic monomers.

European Patent application EP 0 963 761 A1 discloses biomedical devices with coating that are said to be stable, hydrophilic and antimicrobial, and which are formed using a coupling agent, such as a carbodiimide, an acid halide, a chlorosilane, an amino compound, or an isocyanate, to bond a carboxyl-containing hydrophilic coating to the surface by ester or amide linkages.

However, there still is a continued need to provide novel hydrophilic coatings on medical devices for improved biocompatibility. Furthermore, it would be desirable also to provide such coatings on medical devices, such as contact lenses, to allow their use in the human body for an extended period of time. Such a surface-treated lens would be comfortable to wear in actual use and would allow for the extended wear of the lens without irritation or other adverse effects to the cornea. It would be desirable also to provide simple methods for manufacturing such a surface-treated lens.

### SUMMARY OF THE INVENTION

In general, the present invention provides a medical device having a hydrophilic coating.

In one aspect, the hydrophilic coating comprises a polymeric material comprising units of at least a hydrophilic monomer and at least a coupling agent that has coupling functional groups, at least a portion of which is coupled to surface functional groups of the medical device.

In another aspect, the coupling agent is a silane coupling agent that has coupling functional groups that react with at least a portion of the surface functional groups of the medical device.

In another aspect, the medical device comprises silanol groups.

In a further aspect, the medical device comprises alkylsiloxy groups.

In still another aspect, the medical device is pre-treated to increase a population of the surface functional groups.

In still another aspect, the medical devices are ophthalmic devices.

In yet another aspect, the medical devices are contact lenses.

In a further aspect, the present invention provides a method of making a medical device that has a hydrophilic coating. The method comprises: (a) providing the medical device having a plurality of medical-device surface functional groups; (b) providing a coating polymer that comprises units of at least a hydrophilic monomer and a coupling agent that has coupling functional groups capable of interacting with at least a portion of the medical-device surface functional groups; and (c) contacting the medical device with the coating polymer to couple the coating polymer to the surface of the medical device.

In yet another aspect, a method of making a medical device that has a hydrophilic coating comprises: (a) providing the medical device having a plurality of medical-device surface functional groups; (b) providing at least a hydrophilic monomer having a polymerizable functional group and at least a coupling agent having a polymerizable functional group, which coupling agent is capable of interacting with the medical-device surface functional groups; and (c) contacting the medical device with the hydrophilic monomer and the coupling agent to form a hydrophilic coating on the medical device.

In yet another aspect, the method further comprises treating the medical device prior to the step of contacting, to increase a population of the medical-device surface functional groups.

Other features and advantages of the present invention will become apparent from the following detailed description and claims.

### DETAILED DESCRIPTION OF THE INVENTION

In general, the present invention provides a medical device having a hydrophilic coating.

In one aspect, the hydrophilic coating comprises a polymeric material comprising units of at least a hydrophilic monomer and units of at least a coupling agent that has coupling functional groups, at least a portion of which is coupled to surface functional groups of the medical device.

In one aspect, the coupling functional groups and the medical-device surface functional groups are complementary. The term “complementary” means being capable of interacting with one another. Such interacting includes forming a covalent bond, an ionic bond, hydrogen bond, a complexation, or otherwise allowing an attraction between the two components. Preferably, such interacting involves the formation of covalent bonds. Non-limiting examples of such functional groups are alkoxysilanes, halosilanes, vinylsilanes, allylsilanes, aminoalkylsilanes, glycidylsilanes, fluoroalkylsilanes, mercaptoalkylsilanes, carboxysilanes, isocyanatosilanes, and ureidosilanes, preferably interacting with silanol groups, alkoxysilane groups, chlorosilane groups, bromosilane groups, as well as with hydroxyl, sulfhydryl, and carboxy groups. For example, an alkoxysilane group can form a bond with a hydroxyl or silanol group; a glycidyl group with a variety of other functional groups, such as hydroxyl, mercapto, carboxyl, amino, or ureido group; an aminoalkylsilane group with a surface carboxyl group; a carboxylsilane group with a surface hydroxyl or amino group; and a silylisocyanate group with a surface hydroxyl or amino group.

In another aspect, the hydrophilic monomer is selected from the group consisting of nonionic monomers, such as 2-hydroxyethyl methacrylate (“HEMA”), 2-hydroxyethyl acrylate (“HEA”), 2-(2-ethoxyethoxy)ethyl(meth)acrylate, glyceryl(meth)acrylate, polyethylene glycol(meth)acrylate, tetrahydrofurfuryl(meth)acrylate, (meth)acrylamide, N,N′-dimethylmethacrylamide, N,N′-dimethylacrylamide, N-vinyl-2-pyrrolidone, N-vinyl acetamide (or other N-vinyl lactams), and combinations thereof. Other hydrophilic monomers can have more than one polymerizable group, such as tetraethylene glycol(meth)acrylate, triethylene glycol(meth)acrylate, tripropylene glycol(meth)acrylate, ethoxylated bisphenol-A(meth)acrylate, pentaerythritol(meth)acrylate, pentaerythritol(meth)acrylate, ditrimethylolpropane(meth)acrylate, ethoxylated trimethylolpropane(meth)acrylate, dipentaerythritol(meth)acrylate, alkoxylated glyceryl(meth)acrylate. The term “(meth)acrylate” includes acrylate and methacrylate. Similar meanings apply to other analogous terms of “(meth)acrylate.” The hydrophilic monomer can be a hydrophilic prepolymer, such as poly(alkyleneoxy) having varying chain length, functionalized with at least a polymerizable group. Preferably, the free silylhydroxyl group is protected with an alkoxy group, such as a methoxy or ethoxy group. Still further examples of hydrophilic monomers are the vinyl carbonate and vinyl carbamate monomers disclosed in U.S. Pat. No. 5,070,215, and the hydrophilic oxazolone monomers disclosed in U.S. Pat. No. 4,910,277. The contents of these patents are incorporated herein by reference. Other suitable hydrophilic monomers will be apparent to one skilled in the art. Non-limiting examples of polymerizable groups are vinyl, allyl, acryloyl, acryloyloxy, methacryloyl, methacryloyloxy, epoxide, isocyanate, isothiocyanate, amino, hydroxyl, alkoxy, mercapto, anhydride, carboxylic, fumaryl, styryl, itaconyl, maleimido, methacrylamido, acrylamido, and combinations thereof. The hydrophilic monomer also can be an anionic monomer, such as 2-methacryloyloxyethylsulfonate salts. Substituted anionic hydrophilic monomers, such as from acrylic and methacrylic acid, can also be utilized wherein the substituted group can be removed by a facile chemical process. Non-limiting examples of such substituted anionic hydrophilic monomers include trimethylsilyl esters of (meth)acrylic acid, which are hydrolyzed to regenerate an anionic carboxyl group. The hydrophilic monomer also can be a cationic monomer selected from the group consisting of 3-methacrylamidopropyl-N,N,N-trimethyammonium salts, 2-methacryloyloxyethyl-N,N,N-trimethylammonium salts, p-vinylbenzyl-N,N,N-trimethylammonium salts, and amine-containing monomers, such as 3-methacrylamidopropyl-N,N-dimethylamine.

In another aspect, the coupling agent comprises a polymerizable group that is capable of undergoing polymerization with a hydrophilic monomer to from the hydrophilic coating polymer. Alternatively, the monomeric coupling agent comprises a functional group that is capable of reacting with another functional group on a portion of the monomeric units of the coating polymer.

In one embodiment, the coupling agent is selected from the group consisting of silane coupling agents, such as methacryloyloxymethyltrimethoxysilane, methacryloyloxypropyltrimethoxysilane, methacryloyloxymethyltriethoxysilane, methacryloyloxypropyltriethoxysilane, γ-glycidoxypropylmethyldimethoxysilane, γ-glycidoxypropyltrimethoxysilane, γ-glycidoxypropylmethyldiethoxysilane, γ-glycidoxypropyltriethoxysilane, γ-aminopropylmethyldimethoxysilane, γ-aminopropyltrimethoxysilane, γ-aminopropylmethyldimethoxysilane, γ-aminopropyltriethoxysilane, N-aminoethyl-γ-aminopropylmethyldimethoxysilane, N-aminoethyl-γ-aminopropyltrimethoxysilane, N-aminoethyl-γ-aminopropyltriethoxysilane, N-phenyl-γ-aminopropyltrimethoxysilane, N-phenyl-γ-aminopropyltriethoxysilane, N-phenyl-γ-aminopropylmethyldimethoxysilane, N-phenyl-γ-aminopropylmethyldiethoxysilane, γ-mercaptopropylmethyldimethoxysilane, γ-mercaptopropylmethyldiethoxysilane, γ-mercaptopropyltrimethoxysilane, γ-mercaptopropyltriethoxysilane, γ-isocyanatopropylmethyldiethoxysilane, γ-isocyanatopropyltriethoxysilane, vinyltrimethoxysilane, styrylethyltrimethoxysilane, 7-octenyltrimethoxysilane, 10-undecenyltrimethoxysilane, and the like, and they may be used solely or by combinations of two or more of them. These silane coupling agents are commercially available (e.g., from Gelest, Inc., Morrisville, Pa.). It should be understood that although the foregoing silane coupling agents are disclosed having methoxy or ethoxy substituents, other lower alkoxy substituents are equally applicable in the present invention, such as alkoxy groups containing from 1 to, and including, 10 carbon atoms (or from 1 to, and including, 5 carbon atoms, or from 1 to, and including, 3 carbon atoms). A general class of suitable silane coupling agents of the present invention comprises trialkoxysilanes or dialkoxysilanes, each having a polymerizable group. The polymerizable group can be selected from the non-limiting polymerizable groups disclosed above. The mole ratio of the coupling agent to the hydrophilic monomer can be in the range from about 0.001 to about 0.4, or from about 0.01 to about 0.2, or from about 0.01 to about 0.1. It is desirable to include a sufficient number of coupling agent units substantially to react with the population of the medical-device surface functional groups. The number of such surface functional groups can be determined by one skilled in the art using available surface analysis techniques (e.g., XPS or functional group titration). The coating polymer can comprise from about 0.001 to about 20 percent (by weight) (or from about 0.01 to about 10 percent, or from about 0.1 to about 5 percent by weight) of the surface-treated medical device.

Although the preferred medical-device surface functional groups are silanol groups or alkoxysilane groups for forming strong covalent bonds with the coupling functional groups of the coupling agents, the medical-device can include surface functional groups other than silanol groups. In addition, they can be parts of units of the polymeric material of the medical device. For example, hydrogel polymers of contact lenses typically comprise hydrophilic monomeric units, such as 2-hydroxyethyl methacrylate, which provides hydroxyl surface groups. Alternatively, a hydrogel polymer comprising (meth)acrylic acid units has carboxyl surface groups. In still another embodiment, a hydrogel comprising, for example, methacrylamidopropylamine has amino surface groups. In some embodiments, the medical device (e.g., contact lens) material comprises silicone hydrogel, which is a copolymer of siloxy-containing monomers and at least a hydrophilic monomer. The bonds formed between these functional groups and the coupling functional groups of the silane coupling agents can be broken hydrolytically. Under certain circumstances, it can be advantageous to have at least a portion of the coating polymer released in such a manner. For example, a medical-device coating polymer that includes a moiety having a medical value (e.g., therapeutic or diagnostic) can be desirably released hydrolytically at the site of a medical condition that is targeted for treatment. Alternatively, the hydrolytic release of the coating polymer can provide a sustained release oh the hydrophilic polymer that can increase lubricity, increase wetting, and reduce surface deposit.

Other materials suitable for making medical devices in the present invention are disclosed further below.

A non-limiting example of the hydrophilic coating polymer is a copolymer of N,N-dimethylacrylamide and 3-methacryloyloxypropyltrimethoxysilane. This hydrophilic polymer is formed in the following Scheme 1.

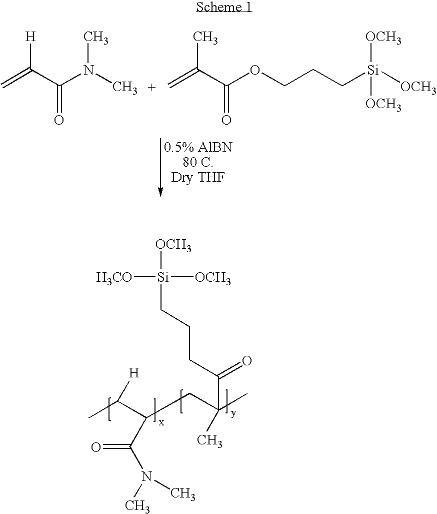

The formation of the hydrophilic coating on exemplary contact lenses is represented in Scheme 2 or 3. 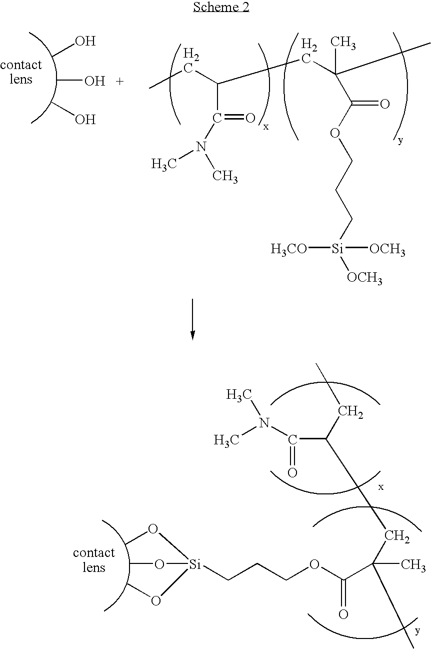 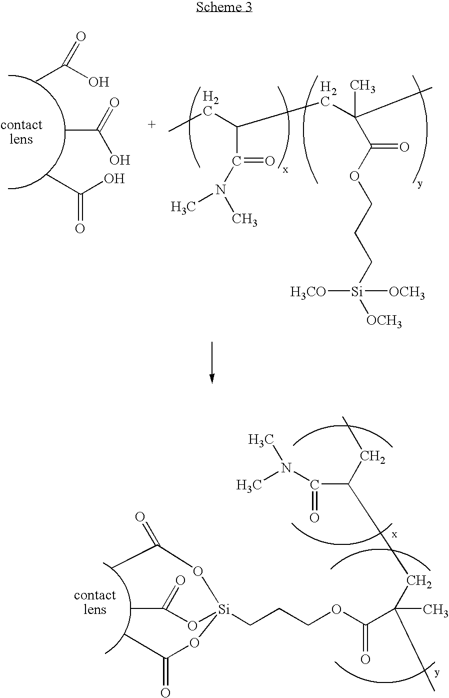
wherein x and y are integers from about 10 to 10,000 (or from about 10 to about 5,000, or from about 10 to about 1,000); and the ratio y/x is in the range from about 0.01 to about 0.4, or from about 0.01 to about 0.2, or from about 0.01 to about 0.1.

In another aspect, a precursor for the coating polymer (“precursor polymer”) comprises units of at least a first monomer that is hydrophilic and at least units of a second monomer that has a reactive functional group. The second monomer also can be hydrophilic. A coupling agent is provided, having coupling functional groups that are capable of interacting with the medical-device surface functional groups and a functional group that is capable of forming a bond with the reactive functional group on the second monomer. The coupling agent is allowed to react with the precursor polymer to produce the coating polymer, which is then applied to the surface of the medical device to form the hydrophilic coating. For example, in one embodiment, the precursor polymer comprises a copolymer of poly(ethylene glycol)methacrylate and methacryloyloxyethylamine. A coupling agent of glycidoxypropyltrimethoxysilane reacts with the amino group of methacryloyloxyethylamine to produce the coating polymer represented by Formula I, which is attached to the surface of a hydrogel contact lens according to the following Scheme 4. 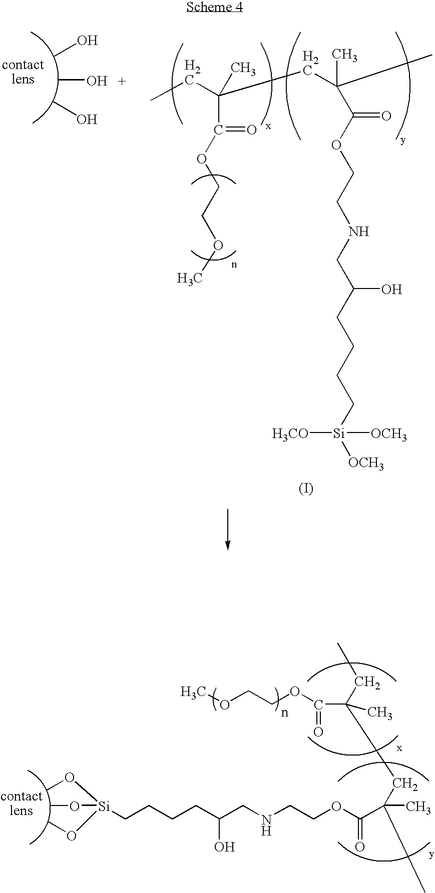
wherein n, x, and y are integers; n is in the range from about 2 to about 50 (or from about 2 to about 20, or from about 5 to about 10); x and y are in the range from about 10 to about 1000 (or from about 10 to 500, or from about 50 to 200); and the ratio y/x is in the range from about 0.01 to about 0.4, or from about 0.01 to about 0.2, or from about 0.01 to about 0.1.

In another embodiment, the hydrophilic coating polymer comprising units of N-vinyl-2-pyrrolidone and units of vinyltrimethoxysilane, as represented by Formula II, wherein x, and y are disclosed above.

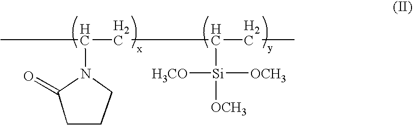

This hydrophilic coating polymer is attached to the surface of the medical device according to the following Scheme 5.

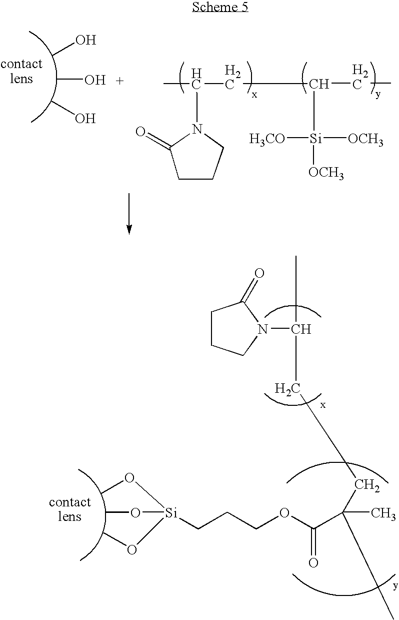

The surface treatment of the medical device can be carried out, for example, at about room temperature or under autoclave condition. The medical device is immersed in a solution comprising the coating polymer. The solution is preferably non-aqueous to prevent internal crosslinking of the alkoxysilane groups. Alternatively, the solution is applied to the surface of the medical device by dipping, spraying, spin coating, or printing.

Monomeric units of the hydrophilic monomer and coupling agent also can be deposited on the surface of the medical device by physical vapor deposition (when they have suitable vapor pressure), and are allowed to polymerize thereon. Such a polymerization may be effected by heat or irradiation.

In another aspect, the surface of the medical device can be treated with a plasma discharge or corona discharge to increase the population of surface groups. The type of gas introduced into the treatment chamber is selected to provide the desired type of surface groups. For example, hydroxyl surface groups can be produced with a treatment chamber atmosphere comprising water vapor or alcohols. Carboxyl surface groups can be generated with a treatment chamber comprising oxygen or air or another oxygen-containing gas. Ammonia or amines in a treatment chamber atmosphere can generate amino surface groups. Sulfur-containing gases, such as organic mercaptans or hydrogen sulfide, can generate mercaptan surface groups. Methods and apparatuses for surface treatment by plasma discharge are disclosed in, for example, U.S. Pat. Nos. 6,550,915 and 6,794,456, which are incorporated herein in their entirety by reference. Methods and apparatuses for corona discharge treatment are also known by people skilled in the art. In one embodiment, the medical device is treated with oxygen-containing plasma, which can be generated in an oxygen-containing atmosphere by a conventional method such as low-pressure electrical discharge, radio-frequency (“RF”) capacitive discharge, RF inductively coupled plasma discharge, microwave-generated plasma discharge, or combinations thereof. A suitable method for the present invention is RF inductively coupled plasma discharge in a low-pressure oxygen-containing atmosphere (such as pressure in the range from about 0.1 Pa to about 1000 Pa), at RF in the range from about 1 MHz to about 100 MHz (such as the commonly used frequency of 13.56 MHz), and at power in the range from about 10 to about 1000 W. The duration of the treatment can be in a range from about 1 second to about 2 hours. Preferably, the treatment duration is sufficient to increase the population of the desired surface functional groups; e.g., from about 10 seconds to about 1 hour.

Medical devices comprising a wide variety of polymeric materials, including hydrogel and non-hydrogel materials, can be made to have improved surface hydrophilicity by a method of the present invention. In general, non-hydrogel materials are hydrophobic polymeric materials that do not contain water in their equilibrium state. Typical non-hydrogel materials comprise silicone acrylates, such as those formed from the bulky siloxy monomer (e.g., tris(trimethylsiloxy)silylpropyl methacrylate, commonly known as “TRIS” monomer), poly(dimethylsiloxy dimethacrylate)prepolymer, or silicones having fluoroalkyl side groups. On the other hand, hydrogel materials comprise hydrated, crosslinked polymeric systems containing water in an equilibrium state. Hydrogel materials contain about 5 weight percent water or more (up to, for example, about 80 weight percent). Non-limiting examples of materials suitable for the manufacture of medical devices, such as contact lenses, are herein disclosed.

Silicone hydrogels generally have a water content greater than about 5 weight percent and more commonly between about 10 to about 80 weight percent. Such materials are usually prepared by polymerizing a mixture containing at least one siloxane-containing monomer, a difunctional macromonomer, and at least one hydrophilic monomer. Typically, either the siloxane-containing macromonomer or a hydrophilic, difunctional monomer functions as a crosslinking agent (a crosslinking agent or crosslinker being defined as a monomer having multiple polymerizable functionalities) or a separate crosslinker may be employed. Applicable siloxane-containing monomeric units for use in the formation of silicone hydrogels are known in the art and numerous examples are provided, for example, in U.S. Pat. Nos. 4,136,250; 4,153,641; 4,740,533; 5,034,461; 5,070,215; 5,260,000; 5,310,779; and 5,358,995, which are incorporated herein by reference.

In a preferred embodiment, the siloxane-containing material provides a sufficient number of device surface silanol groups and the coating polymer is attached to the device surface through these groups. In some other embodiments (e.g., devices including one of the siloxane-containing materials disclosed below), it may be preferred to pretreat the surface of such devices to increase the population of the surface silanol groups, as disclosed above. However, under certain circumstances, it may be desirable to attach the coating polymer to the device surface through surface functional groups other than the silanol group, as disclosed above.

Non-limiting examples of applicable siloxane-containing monomeric units for producing medical devices are now presented. Such exemplary siloxane-containing monomers include bulky polysiloxanylalkyl(meth)acrylic monomers. The term “(meth)acrylic” means methacrylic or acrylic. An example of bulky polysiloxanylalkyl(meth)acrylic monomers are represented by the following Formula III: 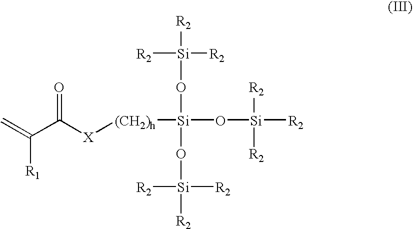
wherein X denotes —O— or —NR—; each R₁ independently denotes hydrogen or methyl; each R₂ independently denotes a lower alkyl radical, phenyl radical or a group represented by 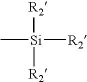
wherein each R′₂ independently denotes a lower alkyl or phenyl radical; and h is from 1 to 10. The term “lower alkyl” means an alkyl radical having 1, 2, 3, 4, 5, 6, 7, 8, 9, or 10 carbon atoms, such as methyl, ethyl, propyl, butyl, isobutyl, pentyl, isopentyl, or hexyl radical.

A suitable bulky monomer is 3-methacryloxypropyltris(trimethyl-siloxy)silane or tris(trimethylsiloxy)silylpropyl methacrylate (“TRIS”).

Another class of representative silicon-containing monomers includes silicone-containing vinyl carbonate or vinyl carbamate monomers such as: 1,3-bis{(4-vinyloxycarbonyloxy)but-1-yl}tetramethyldisiloxane; 3-(trimethylsilyl)propyl vinyl carbonate; 3-(vinyloxycarbonylthio)propyl{tris(trimethylsiloxy)silane}; 3-{tris(trimethylsiloxy)silyl}propyl vinyl carbamate; 3-{tris(trimethylsiloxy)silyl}propyl allyl carbamate; 3-{tris(trimethylsiloxy)silyl}propyl vinyl carbonate; t-butyldimethylsiloxyethyl vinyl carbonate; trimethylsilylethyl vinyl carbonate; and trimethylsilylmethyl vinyl carbonate.

An example of silicon-containing vinyl carbonate or vinyl carbamate monomers are represented by Formula IV: 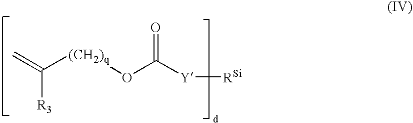
wherein:

Y′ denotes —O—, —S— or —NH—;

R^{Si} denotes a silicon-containing organic radical;

R₃ denotes hydrogen or methyl;

d is 1, 2, 3 or4; and q is 0 or 1.

Suitable silicon-containing organic radicals R^{Si} include the following: 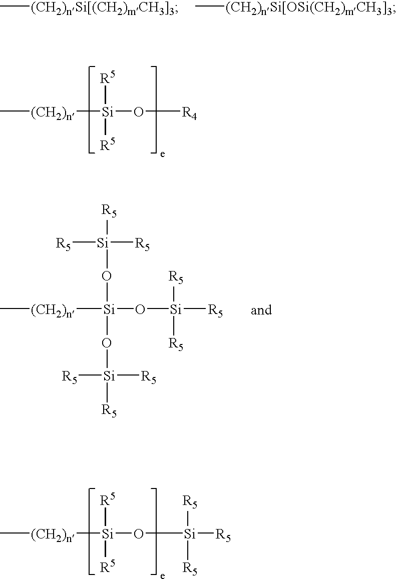
wherein

R₄ denotes 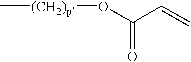
wherein p′ is from 1 to and including 6;

R₅ denotes an alkyl radical or a fluoroalkyl radical having from 1 to and including 6 carbon atoms;

e is 1 to 200; n′ is 1, 2, 3 or 4; and m′ is 0, 1, 2, 3, 4 or 5.

An example of a particular species within Formula IV is represented by Formula V.

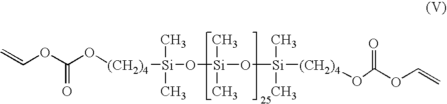

Another class of silicon-containing monomers includes polyurethane-polysiloxane macromonomers (also sometimes referred to as prepolymers), which may have hard-soft-hard blocks like traditional urethane elastomers. They may be end-capped with a hydrophilic monomer such as HEMA. Examples of such silicone urethanes are disclosed in a variety or publications, including Lai, Yu-Chin, “The Role of Bulky Polysiloxanylalkyl Methacryates in Polyurethane-Polysiloxane Hydrogels, ” Journal of Applied Polymer Science, Vol. 60, 1193-1199 (1996). PCT Published Application No. WO 96/31792 discloses examples of such monomers, which disclosure is hereby incorporated by reference in its entirety. Further examples of silicone urethane monomers are represented by Formulae VI and VII:
E(*D*A*D*G)ₐ*D*A*D*E′ (VI)
or
E(*D*G*D*A)ₐ*D*G*D*E′ (VII),
wherein:

D denotes an alkyl diradical, an alkyl cycloalkyl diradical, a cycloalkyl diradical, an aryl diradical or an alkylaryl diradical having 6 to 30 carbon atoms;

G denotes an alkyl diradical, a cycloalkyl diradical, an alkyl cycloalkyl diradical, an aryl diradical or an alkylaryl diradical having 1 to 40 carbon atoms and which may contain ether, thio or amine linkages in the main chain;

* denotes a urethane or ureylene linkage;

a is at least 1;

A denotes a divalent polymeric radical of Formula VIII: 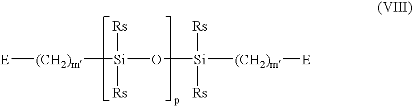
wherein:
each Rₛ independently denotes an alkyl or fluoro-substituted alkyl group having 1 to 10 carbon atoms which may contain ether linkages between carbon atoms;

m′ is at least 1; and

p is a number which provides a moiety weight of 400 to 10,000;
each of E and E′ independently denotes a polymerizable unsaturated organic radical represented by Formula IX: 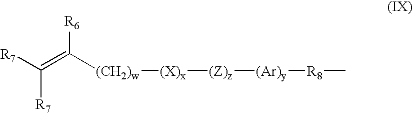
wherein:

R₆ is hydrogen or methyl;

R₇ is hydrogen, an alkyl radical having from 1 to and including 6 carbon atoms, or a —CO—Y—R₉ radical wherein Y is —O—, —S— or —NH—;

R₈ is a divalent alkylene radical having from 1 to and including 10 carbon atoms;

R₉ is a alkyl radical having from 1 to and including 12 carbon atoms;

X denotes —CO— or —OCO—;

Z denotes —O— or —NH—;

Ar denotes a substituted or unsubstituted aromatic radical having from 6 to and including 30 carbon atoms;

w is from 0 to and including 6; x is 0 or 1; y is 0 or 1; and z is 0 or 1.

A more specific example of a silicone-containing urethane monomer is represented by Formula X: 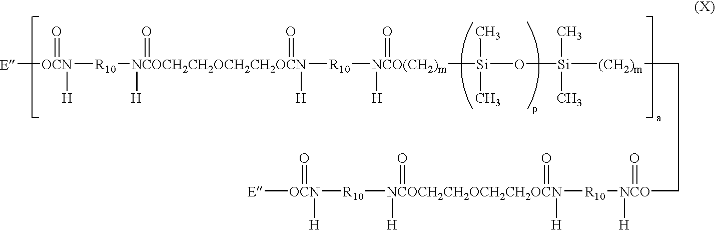
wherein m is at least 1 and is preferably 3 or 4, a is at least 1 and preferably is 1, p is a number which provides a moiety weight of 400 to 10,000 and is preferably at least 30, R₁₀ is a diradical of a diisocyanate after removal of the isocyanate group, such as the diradical of isophorone diisocyanate, and each E″ is a group represented by:

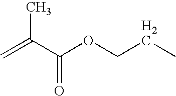

A preferred silicone hydrogel material comprises (in the bulk monomer mixture that is copolymerized) 5 to 50 percent, preferably 10 to 25, by weight of one or more silicone macromonomers, 5 to 75 percent, preferably 30 to 60 percent, by weight of one or more polysiloxanylalkyl(meth)acrylic monomers, and 10 to 50 percent, preferably 20 to 40 percent, by weight of a hydrophilic monomer. In general, the silicone macromonomer is a poly(organosiloxane) capped with an unsaturated group at two or more ends of the molecule. In addition to the end groups in the above structural formulas, U.S. Pat. No. 4,153,641 to Deichert et al. discloses additional unsaturated groups, including acryloxy or methacryloxy. Fumarate-containing materials such as those taught in U.S. Pat. Nos. 5,512,205; 5,449,729; and 5,310,779 to Lai are also useful substrates in accordance with the invention. These patents are incorporated herein by reference. Preferably, the silane macromonomer is a silicon-containing vinyl carbonate or vinyl carbamate or a polyurethane-polysiloxane having one or more hard-soft-hard blocks and end-capped with a hydrophilic monomer.

In particular regard to contact lenses, the fluorination of certain monomers used in the formation of silicone hydrogels has been indicated to reduce the accumulation of deposits on contact lenses made therefrom, as described in U.S. Pat. Nos. 4,954,587, 5,079,319 and 5,010,141, which are incorporated herein by reference. Moreover, the use of silicone-containing monomers having certain fluorinated side groups (e.g., —(CF₂)—H) have been found to improve compatibility between the hydrophilic and silicone-containing monomeric units, as described in U.S. Pat. Nos. 5,387,662 and 5,321,108.

Solvents useful in the surface treatment of the medical device, such as a contact lens, include solvents that readily solubilize the polymers such as carboxylic acids, sulfonic acids, fumaric acid, maleic acids, anhydrides such as maleic anhydride, and functionalized alcohols such as vinyl alcohol. Suitable solvents include tetrahydrofuran (“THF”), acetonitrile, and N,N-dimethyl formamide (“DMF”).

The present invention also provides a method for making a medical device that has a hydrophilic coating. The method comprises: (a) providing the medical device having a plurality of medical-device surface functional groups; (b) providing a coating polymer that comprises units of at least a hydrophilic monomer and units of a coupling agent that has coupling functional groups capable of interacting with at least a portion of the medical-device surface functional groups; and (c) contacting the medical device with the coating polymer to couple the coating polymer to the surface of the medical device. The step of contacting can be carried out, for example, at about room temperature or under autoclave condition for a sufficient time to react substantially all of the surface groups with the coupling groups of the coating polymer, for example, for a time from about 1 minute to about 10 hours (or from about 1 minute to about 5 hours, or from about 5 minutes to about 1 hour). Coating polymer molecules that have not been attached to the surface of the medical device can then be removed by rinsing.

In yet another aspect, the present invention provides a method of making a medical device that has a hydrophilic coating. The method comprises: (a) providing the medical device having a plurality of medical-device surface functional groups; (b) providing at least a hydrophilic monomer having a polymerizable functional group and at least a coupling agent having a polymerizable functional group and at least a coupling functional group, which is capable of interacting with the medical-device surface functional groups; and (c) contacting the medical device with the hydrophilic monomer and the coupling agent to form a hydrophilic coating on the medical device. In one embodiment, the medical device is contacted with the hydrophilic monomer and the coupling agent simultaneously. Coating polymer molecules that have not been attached to the surface of the medical device can then be removed by rinsing.

Alternatively, a method of making a medical device that has a hydrophilic coating comprises: (a) contacting the medical device having a plurality of medical-device surface functional groups with at least a coupling agent having at least a coupling functional group and another reactive functional group, said coupling functional group being capable of interacting with the medical-device surface functional groups, to form a medical device having surface coupling agent; (b) providing a hydrophilic coating polymer comprising units of at least a hydrophilic monomer and a second monomer that has a reactive functional group that is complementary to said another reactive functional group of said coupling agent; and (c) contacting the medical device having surface coupling agent with the hydrophilic coating polymer to form the hydrophilic coating on the medical device. Coating polymer molecules that have not been attached to the surface of the medical device can then be removed by rinsing.

In a further aspect, the population of the medical-device surface groups can be increased by a treatment method, such as corona discharge or plasma discharge, as disclosed above, prior to immersing the medical device in the solution containing the coating polymer. Alternatively, the population of the medical-device surface groups can be increased by a wet treatment method, such as an acid treatment.

In one embodiment, the coupling agent of any of the methods disclosed above is a silane coupling agent.

Ophthalmic medical devices manufactured to have a hydrophilic surface coating of the present invention are used as customary in the field of ophthalmology. For example, in a surgical cataract procedure, an incision is placed in the cornea of an eye. Through the corneal incision the cataractous natural lens of the eye is removed (aphakic application) and an IOL is inserted into the anterior chamber, posterior chamber or lens capsule of the eye prior to closing the incision. However, the subject ophthalmic devices may likewise be used in accordance with other surgical procedures known to those skilled in the field of ophthalmology.

In some embodiments, the surface-treated medical devices include, without limitation, stents, implants, catheters, and ophthalmic devices.

### EXAMPLE 1

### Synthesis of a Copolymer of N,N-Dimethylacrylamide (“DMA”) and 3-(Methacryloyloxypropyl)trimethoxysilane (“MTS”)

To a one-liter round bottom flask under dry nitrogen are added 50 g of DMA, 50 g of MTS, 0.5 g of Vazo 64™ (a thermal polymerization initiator, said to be 2,2′-azobisisobutyronitrile, DuPont Chemical Company, Wilmington, Del.), and 500 ml of freshly distilled tetrahydrofuran (“THF”). The reaction mixture is heated to 80° C. for four hours, at which time the reaction solution is devolatilized under fine vacuum to remove THF, resulting in a quantitative yield of DMA-MTS copolymer.

### EXAMPLE 2

### Plasma Treatment of Pure Vision™ Lenses

Pure Vision™ silicone hydrogel lenses (from Bausch and Lomb Incorporated, Rochester, N.Y.) are plasma treated with air plasma at a treatment time of about 16 minutes in a Metroline/IPC 7104 radio-frequency (“RF”) plasma chamber at pressure of about 40 Pa (or 0.3 mm Hg), power of 400 W, and RF of 13.56 MHz.

### EXAMPLE 3

### Coating of Plasma-Treated Pure Vision™ Lenses

Plasma-treated Pure Vision™ lenses of Example 2 are placed in vials together with the coating solution (1% DMA-MTS copolymer in dry acetonitrile) and kept at about 60° C. for one hour. The lenses are then removed from the vials and rinsed several times with purified water to yield lenses having a coating of poly(DMA-co-MTS). Coated lenses are placed back in the vials with 5 ml of phosphate buffer saline and autoclaved for sterilization.

### EXAMPLE 4

### Acid-Treated Fumarate 36 Lenses

Fumarate 36 lenses (developmental lenses, see U.S. Pat. No. 6,213,604) comprising a silicone fumarate prepolymer, DMA, and TRIS are treated in aqueous 0.1 N solution of hydrochloric acid at about 60° C., for a time in the range from about 1 minute to about 1 hour (preferably, from about 1 minute to about 15 minutes). The treatment is to increase the population of lens surface silanol groups. Then, the lenses are removed from the solution and rinsed several times with purified water. Excess water is removed from the lenses.

### EXAMPLE 5

### Coating of Acid-Treated Fumarate 36 Lenses

Each acid-treated Fumarate 36 lens of Example 4 is placed in a vial with about 3 ml of a solution formulated from 5 g of the poly(DMA-co-MTS) of Example 1 and 100 ml of dry acetonitrile, and kept at 60° C. for about 2 hours. The treatment yield lenses having a hydrophilic coating of poly(DMA-co-MTS). The coated lenses are removed from the solution and rinsed several times with purified water.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A surface-treated medical device comprising a hydrophilic coating polymer that comprises units of at least a hydrophilic monomer and units of a silane coupling agent, wherein an untreated medical device has a plurality of medical-device surface functional groups, and the silane coupling agent has at least a coupling functional group that is capable of interacting with the medical-device surface functional groups.

2. The surface-treated medical device of claim 1, wherein the hydrophilic coating polymer is a copolymer of the hydrophilic monomer and the silane coupling agent.

3. The surface-treated medical device of claim 1, wherein the plurality of medical-device surface functional groups and the coupling functional group are complementary and are selected from the group consisting of alkoxysilanes, halosilanes, vinylsilanes, allylsilanes, aminoalkylsilanes, glycidylsilanes, fluoroalkylsilanes, mercaptoalkylsilanes, carboxysilanes, isocyanatosilanes, ureidosilanes, hydroxyl, alkoxy, glycidyl, mercapto, carboxyl, amino, isocyanate, and ureido.

4. The surface-treated medical device of claim 1, wherein the silane coupling agent is selected from the group consisting of trialkoxysilanes and dialkoxysilanes, each having a polymerizable group.

5. The surface-treated medical device of claim 4, wherein the alkoxy group comprises from 1 to, and including, 10 carbon atoms.

6. The surface-treated medical device of claim 1, wherein the silane coupling agent is selected from the group consisting of methacryloyloxymethyltrimethoxysilane, methacryloyloxypropyltrimethoxysilane, methacryloyloxymethyltriethoxysilane, methacryloyloxypropyltriethoxysilane, γ-glycidoxypropylmethyldimethoxysilane, γ-glycidoxypropyltrimethoxysilane, γ-glycidoxypropylmethyldiethoxysilane, γ-glycidoxypropyltriethoxysilane, γ-aminopropylmethyldimethoxysilane, γ-aminopropyltrimethoxysilane, γ-aminopropylmethyldimethoxysilane, γ-aminopropyltriethoxysilane, N-aminoethyl-γ-aminopropylmethyldimethoxysilane, N-aminoethyl-γ-aminopropyltrimethoxysilane, N-aminoethyl-γ-aminopropyltriethoxysilane, N-phenyl-γ-aminopropyltrimethoxysilane, N-phenyl-γ-aminopropyltriethoxysilane, N-phenyl-γ-aminopropylmethyldimethoxysilane, N-phenyl-γ-aminopropylmethyldiethoxysilane, γ-mercaptopropylmethyldimethoxysilane, γ-mercaptopropylmethyldiethoxysilane, γ-mercaptopropyltrimethoxysilane, γ-mercaptopropyltriethoxysilane, γ-isocyanatopropylmethyldiethoxysilane, γ-isocyanatopropyltriethoxysilane, vinyltrimethoxysilane, styrylethyltrimethoxysilane, 7-octenyltrimethoxysilane, 10-undecenyltrimethoxysilane, and combinations thereof.

7. The surface-treated medical device of claim 1, wherein the hydrophilic monomer is selected from the group consisting of 2-hydroxyethyl(meth)acrylate, 2-(2-ethoxyethoxy)ethyl(meth)acrylate, glyceryl(meth)acrylate, tetrahydrofurfuryl(meth)acrylate, (meth)acrylamide, N,N′-dimethyl(meth)acrylamide, N-vinylacetamide, N-vinyl lactams, tetraethylene glycol(meth)acrylate, triethylene glycol(meth)acrylate, tripropylene glycol(meth)acrylate, ethoxylated bisphenol-A(meth)acrylate, pentaerythritol(meth)acrylate, pentaerythritol(meth)acrylate, ditrimethylolpropane(meth)acrylate, ethoxylated trimethylolpropane(meth)acrylate, dipentaerythritol(meth)acrylate, alkoxylated glyceryl(meth)acrylate, poly(alkyleneoxy) having varying chain length, functionalized with at least a polymerizable group, vinyl carbonate, vinyl carbamate, 3-methacrylamidopropyl-N,N,N-trimethyammonium salts, 2-methacryloyloxyethyl-N,N,N-trimethylammonium salts, 2-methacryloyloxyethylsulfonate salts, and combinations thereof.

8. The surface-treated medical device of claim 1, wherein each of the hydrophilic monomer and the silane coupling agent has a polymerizable group that is selected from the group consisting of vinyl, allyl, acryloyl, acryloyloxy, methacryloyl, methacryloyloxy, epoxide, isocyanate, isothiocyanate, amino, hydroxyl, alkoxy, mercapto, anhydride, carboxylic, fumaryl, styryl, itaconyl, maleimido, methacrylamido, acrylamido, and combinations thereof.

9. The surface-treated medical device of claim 8, wherein each of the hydrophilic monomer and the silane coupling agent has a polymerizable group that is selected from the group consisting of acryloyl, acryloyloxy, methacryloyl, methacryloyloxy, acrylamido, metahcrylamido, and combinations thereof.

10. The surface-treated medical device of claim 1, wherein the medical device comprises silicone hydrogel.

11. The surface-treated medical device of claim 10, wherein the medical-device surface functional groups are hydroxyl groups and the coupling functional group is an alkoxy group.

12. The surface-treated medical device of claim 10, wherein the medical device is a contact lens.

13. The surface-treated medical device of claim 1, wherein the medical device is selected from the group consisting of stents, implants, catheters, and ophthalmic devices.

14. A surface-treated medical device comprising a hydrophilic coating polymer that comprises units of at least a hydrophilic monomer and units of a silane coupling agent, each having a polymerizable group; wherein an untreated medical device has a plurality of medical-device surface functional groups, and the silane coupling agent has at least a coupling functional group that is capable of interacting with the medical-device surface functional groups; the medical-device surface functional groups and the coupling functional group are complementary and are selected from the group consisting of alkoxysilanes, halosilanes, vinylsilanes, allylsilanes, aminoalkylsilanes, glycidylsilanes, fluoroalkylsilanes, mercaptoalkylsilanes, carboxysilanes, isocyanatosilanes, ureidosilanes, hydroxyl, alkoxy, glycidyl, mercapto, carboxyl, amino, isocyanate, and ureido; and the polymerizable group is selected from the group consisting of vinyl, allyl, acryloyl, acryloyloxy, methacryloyl, methacryloyloxy, epoxide, isocyanate, isothiocyanate, amino, hydroxyl, alkoxy, mercapto, anhydride, carboxylic, fumaryl, styryl, itaconyl, maleimido, methacrylamido, acrylamido, and combinations thereof.

15. The surface-treated medical device of claim 14, wherein the medical device comprises silicone hydrogel.

16. The surface-treated medical device of claim 14, wherein the medical device is a contact lens.

17. A polymer comprising at least units of a hydrophilic monomer and units of a silane coupling agent having at least a free coupling functional group.

18. The polymer of claim 17, wherein each of the hydrophilic monomer and the silane coupling agent has at least a polymerizable functional group selected from the group consisting of vinyl, allyl, acryloyl, acryloyloxy, methacryloyl, methacryloyloxy, epoxide, isocyanate, isothiocyanate, amino, hydroxyl, alkoxy, mercapto, anhydride, carboxylic, fumaryl, styryl, itaconyl, maleimido, methacrylamido, acrylamido, and combinations thereof.

19. The polymer of claim 17, wherein each of the hydrophilic monomer and the silane coupling agent has at least a polymerizable functional group selected from the group consisting of acryloyl, acryloyloxy, methacryloyl, methacryloyloxy, and combinations thereof.

20. The polymer of claim 17, comprising a copolymer of the hydrophilic monomer and the silane coupling agent.

21. The polymer of claim 17, further comprising an additional monomer having an additional-monomer reactive functional group; wherein the polymer is a copolymer of the hydrophilic monomer and the additional monomer, and the silane coupling agent is attached to the polymer via the additional-monomer reactive functional group.

22. The polymer of claim 17, wherein the hydrophilic monomer is selected from the group consisting of nonionic monomers, cationic monomers, and anionic monomers.

23. The polymer of claim 17, wherein the hydrophilic monomer is selected from the group consisting of 2-hydroxyethyl(meth)acrylate, 2-(2-ethoxyethoxy)ethyl(meth)acrylate, glyceryl(meth)acrylate, tetrahydrofurfuryl(meth)acrylate, (meth)acrylamide, N,N′-dimethyl(meth)acrylamide, N-vinyl lactams, N-vinyl acetamide, tetraethylene glycol(meth)acrylate, triethylene glycol(meth)acrylate, tripropylene glycol(meth)acrylate, ethoxylated bisphenol-A(meth)acrylate, pentaerythritol(meth)acrylate, pentaerythritol(meth)acrylate, ditrimethylolpropane(meth)acrylate, ethoxylated trimethylolpropane(meth)acrylate, dipentaerythritol(meth)acrylate, alkoxylated glyceryl(meth)acrylate, poly(alkyleneoxy) having varying chain length, functionalized with at least a polymerizable group, vinyl carbonate, vinyl carbamate, 3-methacrylamidopropyl-N,N,N-trimethyammonium salts, 2-methacryloyloxyethyl-N,N,N-trimethylammonium salts, 2-methacryloyloxyethylsulfonate salts, and combinations thereof.

24. The polymer of claim 17, wherein the silane coupling agent is selected from the group consisting of methacryloyloxymethyltrimethoxysilane, methacryloyloxypropyltrimethoxysilane, methacryloyloxymethyltriethoxysilane, methacryloyloxypropyltriethoxysilane, γ-glycidoxypropylmethyldimethoxysilane, γ-glycidoxypropyltrimethoxysilane, γ-glycidoxypropylmethyldiethoxysilane, γ-glycidoxypropyltriethoxysilane, γ-aminopropylmethyldimethoxysilane, γ-aminopropyltrimethoxysilane, γ-aminopropylmethyldimethoxysilane, γ-aminopropyltriethoxysilane, N-aminoethyl-γ-aminopropylmethyldimethoxysilane, N-aminoethyl-γ-aminopropyltrimethoxysilane, N-aminoethyl-γ-aminopropyltriethoxysilane, N-phenyl-γ-aminopropyltrimethoxysilane, N-phenyl-γ-aminopropyltriethoxysilane, N-phenyl-γ-aminopropylmethyldimethoxysilane, N-phenyl-γ-aminopropylmethyldiethoxysilane, γ-mercaptopropylmethyldimethoxysilane, γ-mercaptopropylmethyldiethoxysilane, γ-mercaptopropyltrimethoxysilane, γ-mercaptopropyltriethoxysilane, γ-isocyanatopropylmethyldiethoxysilane, γ-isocyanatopropyltriethoxysilane, vinyltrimethoxysilane, styrylethyltrimethoxysilane, 7-octenyltrimethoxysilane, 10-undecenyltrimethoxysilane, and combinations thereof.

25. A method for making a medical device that has a hydrophilic coating, said method comprising:
(a) providing the medical device having a plurality of medical-device surface functional groups;
(b) providing a coating polymer that comprises units of at least a hydrophilic monomer and units of a silane coupling agent that has coupling functional groups capable of interacting with at least a portion of the medical-device surface functional groups; and
(c) contacting the medical device with the coating polymer to couple the coating polymer to the surface of the medical device.

26. The method of claim 25, further comprising the step of treating the medical device to increase a population of the medical-device surface functional groups.

27. The method of claim 26, wherein the step of treating the medical device comprises exposing the medical device to a corona discharge or a plasma discharge treatment.

28. A method for making a medical device that has a hydrophilic coating, said method comprising:
(a) providing the medical device having a plurality of medical-device surface functional groups;
(b) providing at least a hydrophilic monomer having a polymerizable group and at least a silane coupling agent having a polymerizable group; and
(c) contacting the medical device with the hydrophilic monomer and the silane coupling agent simultaneously to form the hydrophilic coating.

29. The method of claim 29, further comprising the step of treating the medical device to increase a population of the medical-device surface functional groups.

30. The method of claim 31, wherein the step of treating the medical device comprises exposing the medical device to a corona discharge or a plasma discharge treatment.

31. A method for making a medical device that has a hydrophilic coating, said method comprising:
(a) contacting the medical device having a plurality of medical-device surface functional groups with at least a silane coupling agent having at least a coupling functional group and at least a first reactive functional group, the coupling functional group being capable of interacting with the medical-device surface functional groups, producing the medical device having attached coupling agent;
(b) providing a hydrophilic coating polymer comprising units of at least a hydrophilic monomer and a second monomer that has a second reactive functional group that is capable of reacting with the first reactive functional group; and
(c) contacting the medical device having attached coupling agent with the hydrophilic coating polymer to form the hydrophilic coating on the medical device.

32. The method of claim 31, further comprising the step of treating the medical device to increase a population of the medical-device surface functional groups.

33. The method of claim 35, wherein the step of treating the medical device comprises exposing the medical device to a corona discharge or a plasma discharge treatment.
